(19) United States
(12) Patent Application Publication
(10) Pub. No.: US 2026/0098843 A1
(43) Pub. Date: Apr. 9, 2026

(54) GAS DETECTION SENSOR ARRANGEMENT AND METHOD FOR ASSEMBLY

(71) Applicant: Danfoss A/S, Nordborg (DK)

(72) Inventors: Michael Taszarek, Sønderborg (DK); Ingvar Smari Kampp, Nordborg (DK); Veenith Shetty, Sønderborg (DK); Apoorva Tyagi, Sønderborg (DK); Mohamed Benslimane, Nordborg (DK); Minh Khoa Tran Nguyen, Nordborg (DK); Wayne E. Alphonso, Nordborg (DK)

(21) Appl. No.: 18/907,924

(22) Filed: Oct. 7, 2024

(51) Int. Cl. G01N 33/00

(52) U.S. Cl. CPC G01N 33/0027

(57) ABSTRACT

A gas detection sensor arrangement includes an enclosure having a top portion, a bottom portion, an electrical connector, and a circuit board and a gas sensor disposed within the enclosure. The gas sensor is configured to detect gas in an environment external to the enclosure. The bottom portion, the top portion and the electrical connector are joined together to seal the circuit board within the enclosure. A method for assembling the gas detection sensor arrangement includes joining the bottom portion and the top portion together, biasing the gas sensor toward a gas sensor opening formed in the enclosure, and sealing the circuit board within the enclosure.

### TECHNICAL FIELD

The disclosure relates to the field of gas detection sensors for detecting leaks in air conditioning systems, refrigeration systems, furnaces or other combustion systems, heat pumps, etc. This disclosure also relates to the field of gas sensor enclosures provided to protect the gas detection sensors and associated electronics from detrimental external conditions.

### BACKGROUND

When HVAC or other refrigeration systems use refrigerants exhibiting lower global warming potential (GWP), flammability or toxicity hazards may occur in case of refrigerant leak. This is especially true when using flammable refrigerants (A3), for example, R290 or R600a. This is also true even when using lower toxic or mildly flammable (A2L) refrigerants, for example, R32 or R1234ze/yf, or blends such as R454B, as such mildly flammable refrigerants have an increased potential to burn as their concentration increases. Thus, the incorporation of a refrigerant leak detection mechanism into such systems has become mandatory for safety reasons.

For gas leak detection sensors to function properly, the sensors should be enclosed in a housing unit so the sensor and its components can be protected against harsh conditions. The current industry solution for protecting gas leak detection sensors utilizes covers and fasteners to secure gas leak detection sensors.

### SUMMARY

According to a first aspect, a gas detection sensor arrangement includes an enclosure having a top portion and a bottom portion, a circuit board disposed within the enclosure, and a gas sensor disposed within the enclosure. The gas sensor is configured to detect gas in an environment external to the enclosure. The bottom portion and the top portion are joined together to seal the circuit board within the enclosure.

The bottom portion and the top portion may be welded together (e.g., via ultrasonic welding or laser welding). Optionally, the bottom portion and the top portion may be bonded together using an adhesive bond (e.g., via epoxy or potted together using a silicone filler, one-or two-part silicone). Even further, the bottom portion and the top portion may be mechanically fastened to each other using, for example, screws or complementary snap-fit features. A seal or sealing element, e.g., O-ring gasket, UV-cured adhesive, relatively soft polymer, etc., may be provided between the bottom portion and the top portion when the bottom portion and the top portion are joined via mechanical means.

The enclosure may further include an electrical connector opening and an electrical connector having an electrical connector adapter flange. The electrical connector opening and the electrical connector adapter flange have opposing surfaces that are joined together to seal the electrical connector opening.

The electrical connector opening and the electrical connector adapter flange may be welded together (e.g., via ultrasonic welding or laser welding). Optionally, the electrical connector opening and the electrical connector adapter flange may be bonded together using an adhesive bond (e.g., via UV-cured adhesive or an epoxy or potted together using a silicone filler, one-or two-part silicone). Even further, the electrical connector and the enclosure may be mechanically fastened to each other using, for example, screws or complementary snap-fit features. A seal or sealing element, e.g., O-ring gasket, UV-cured adhesive, relatively soft polymer, etc., may be provided between the electrical connector opening and the electrical connector adapter flange when the electrical connector opening and the electrical connector adapter flange are joined via mechanical means.

The enclosure may further include a gas sensor opening configured to allow gas in an environment external to the enclosure to reach the gas sensor. The gas sensor opening may be configured as a through hole in a bottom plate of the bottom portion. According to a preferred embodiment, the gas sensor opening may be configured as a simple through hole in a flat portion of the bottom plate of the bottom portion, i.e., without any standoff or spacer portion provided around the through hole within the enclosure. The enclosure is configured to bias the gas sensor towards the gas sensor opening to thereby seal the gas sensor opening.

One or more microcontrollers, relays, switches and/or other electronic components may be disposed on a first side and/or a second side of the circuit board, wherein the first side of the circuit board faces the top portion and the second side of the circuit board faces the bottom portion.

The gas detection sensor arrangement may further include a seal or sealing member, e.g., O-ring gasket, relatively soft polymer, etc., located between the gas sensor and the gas sensor opening. The enclosure may bias or force the gas sensor towards the gas sensor opening, thereby compressing the sealing member between the gas sensor and the enclosure and sealing the gas sensor opening.

The gas detection sensor arrangement may further include a light guide that seals a light guide opening formed in the enclosure. For example, the light guide may emit light from a light source, such as a PCB mounted LED, typically in red or green color light. Alternatively, the enclosure may be provided with a transparent or translucent (or any other color that allows light transmission therethrough) wall portion configured to allow light from a light source located within the enclosure to be visible external to the enclosure. For example, the light guide opening or the transparent or translucent wall portion may be provided in the bottom portion of the enclosure to thereby allow light to pass through the bottom portion.

According to another aspect, components for an enclosure for a gas detection sensor arrangement are provided. The components include a top portion of the enclosure and a bottom portion of the enclosure. At least one of the top portion or the bottom portion is configured to receive a circuit board. At least one of the top portion or the bottom portion is configured to receive a gas sensor configured to detect a gas in an environment external to the enclosure. At least one of the top portion or the bottom portion includes a gas sensor opening configured to allow gas in the environment external to the enclosure to reach the gas sensor. The top portion and the bottom portion are configured to be joined together to seal the circuit board and the gas sensor within the enclosure.

According to a preferred embodiment, opposing surfaces of the top portion and the bottom portion are configured to be ultrasonically welded together at an enclosure seam. At least one opposing surface of the top portion or the bottom portion may have an energy director located thereon. The energy director is configured to initially space the opposing surfaces of the top portion and the bottom portion apart at the enclosure seam and to at least partially melt under an application of energy to the enclosure seam such that the opposing surfaces of the top portion and the bottom portion are brought together and ultrasonically welded together.

Alternatively, opposing surfaces of the top portion and the bottom portion may be joined via the use of laser welding. At least one of the top portion and the bottom portion may be provided with a material that transmits the laser beam energy, thereby facilitating the ability of the laser beam to impinge upon and melt the opposing surface of the other of the top portion and the bottom portion. At least one of the top portion and the bottom portion may be provided with a material that absorbs the laser beam energy, thereby facilitating the ability of the laser beam to melt this absorptive material. Typically, one of the top portion or the bottom portion will be provided with a material that transmits the laser beam energy and the other of the top portion or the bottom portion will be provided with a material that absorbs the laser beam energy.

As another alternative, the top portion and the bottom portion may be joined with mechanical fasteners (e.g., screws, complementary snap-fit features), and/or adhesive bonding (e.g., UV-cured adhesive, epoxy, potting fillers, etc.). When mechanical fasteners are used, a sealing element may additionally be provided between the opposing surfaces. As an even other alternative, a sealant may be applied on the assembled portions of the housing unit, e.g., joint or gap between cover portion and the bottom portion, electrical connector and housing unit portions, and/or gas sensor and any housing unit portions.

The components may further include an electrical connector having an electrical connector adapter flange. At least one of the top portion or the bottom portion includes an electrical connector opening. According to a preferred embodiment, opposing surfaces of the electrical connector adapter flange and the electrical connector opening are configured to be ultrasonically welded together to seal the electrical connector opening. At least one of the opposing surfaces of the electrical connector adapter flange and the electrical connector opening may have an energy director located between the opposing surfaces. The energy director is configured to initially space the opposing surfaces of the electrical connector adapter flange and the electrical connector opening apart, and is further configured to at least partially melt under an application of energy to the opposing surfaces such that the opposing surfaces of the electrical connector adapter flange and the electrical connector opening may be subsequently brought together and ultrasonically welded together.

Alternatively, opposing surfaces of the connector and the electrical connector opening may be joined via the use of laser welding. At least one of the electrical connector adapter flange and the electrical connector opening may be provided with a material that transmits the laser beam energy, thereby facilitating the ability of the laser beam to impinge upon and melt the opposing surface of the other of the electrical connector adapter flange and the electrical connector opening. At least one of the electrical connector adapter flange and the electrical connector opening may be provided with a material that absorbs the laser beam energy, thereby facilitating the ability of the laser beam to melt this absorptive material. Typically, one of the electrical connector adapter flange and the electrical connector opening will be provided with a material that transmits the laser beam energy and the other of t the electrical connector adapter flange and the electrical connector opening will be provided with a material that absorbs the laser beam energy.

As another alternative, the electrical connector adapter flange and the electrical connector opening may be joined using mechanical fasteners (e.g., screws, complementary snap-fit features), and/or adhesive bonding (e.g., UV-cured adhesive, epoxy, potting fillers, etc.). When mechanical fasteners are used, a sealing element may additionally be provided between the opposing surfaces.

The gas sensor opening may be configured to be sealed, at least in part, by the gas sensor. The components may further include a sealing member configured to be disposed between the gas sensor and at least one of the top portion or the bottom portion and configured to, at least in part, seal the gas sensor opening. Alternatively, a sealant may be provided on the top portion or the bottom portion, for example, on an exterior surface around the perimeter of the gas sensor opening, in order to seal, at least in part, the gas sensor opening. The gas sensor opening may be contoured to facilitate placement and retention of the sealant.

According to certain aspects, a method for assembling a gas detection sensor arrangement is provided. The method includes providing a first portion of an enclosure for the gas detection sensor arrangement, providing a second portion of the enclosure for the gas detection sensor arrangement, and providing an electrical connector having an electrical connector adapter flange. The method further includes joining the electrical connector adapter flange to an electrical connector opening provided in one of the first portion or the second portion, assembling a circuit board having a gas sensor disposed thereon to the electrical connector, and joining the first portion to the second portion at an enclosure seam to seal the circuit board inside the enclosure.

The joining method may include welding (ultrasonic or laser), mechanical fasteners e.g., screws, complementary snap-fit features, etc.), and/or adhesive bonding (e.g., UV-cured adhesive, epoxy, potting fillers, etc.).

The method may further include placing a sealing member between the gas sensor and one of the first portion or the second portion and compressing the sealing member between the gas sensor and the one of the first portion or the second portion during the step of joining (e.g., welding, fastening, bonding, etc.) the first portion to the second portion. The step of placing the sealing member between the gas sensor and the one of the first portion or the second portion may include placing the sealing member around a gas sensor opening formed in the one of the first portion or the second portion and compressing the sealing member between the gas sensor and the one of the first portion or the second portion. During the step of joining the first portion to the second portion, the sealing member may seal the gas sensor opening.

The step of ultrasonically welding the first portion to the second portion may include providing an energy director between opposing surfaces of the first portion and the second portion, at least partially melting the energy director, and bringing the opposing surfaces of the first portion and the second portion together at the enclosure seam as the energy director melts.

Similarly, the step of ultrasonically welding the electrical connector adapter flange to the electrical connector opening may include providing an energy director between opposing surfaces of the electrical connector adapter flange and the electrical connector opening, at least partially melting the energy director, and bringing the opposing surfaces of the electrical connector adapter flange and the electrical connector opening together as the energy director melts.

The sealed enclosure may protect the sensor and other components within the enclosure from moisture, refrigerant oils, mechanical forces, UV light, corrosion, particles, and harsh thermal conditions. The enclosure can be particularly advantageous because it protects the gas detection sensor arrangement from frost formation, which occurs during operation, and from pressurized water jets, which are often used to clean gas sensors.

### BRIEF DESCRIPTION OF THE DRAWINGS

Example embodiments of the present disclosure will now be illustrated with reference to the following Figures.

FIG. 1 is a top front perspective view of an embodiment of a gas detection sensor arrangement with a six-pin connector.

FIG. 2 is a partially exploded, top front perspective view of the gas detection sensor arrangement of FIG. 1.

FIG. 3 is an exploded view from a top front perspective of the gas detection sensor arrangement of FIG. 1.

FIG. 4 is a partially exploded, bottom perspective view of the gas detection sensor arrangement of FIG. 1, with the PCBA and the bottom portion removed.

FIG. 5 is a bottom perspective view of the gas detection sensor arrangement of FIG. 1, with the bottom portion removed.

FIG. 6 is a bottom perspective view of the gas detection sensor arrangement of FIG. 1.

FIG. 7 is a schematic side view of an embodiment of a gas detection sensor arrangement showing energy directors.

FIG. 8 is a partially exploded, bottom perspective view of a gas detection sensor arrangement according to an alternative embodiment.

FIG. 9 is a bottom perspective view of a gas detection sensor arrangement according to a further embodiment.

FIG. 10 is an exploded, bottom perspective view of a gas detection sensor arrangement, with the PCBA and the bottom portion removed, according to another embodiment.

FIG. 11(a) is an enlarged detail from FIG. 7 showing an energy director at an ultrasonically welded seam between a top portion and a bottom portion of the enclosure. FIG. 11(b) is an alternative embodiment of the enlarged detail from FIG. 7 showing a laser welded seam between a top portion and a bottom portion of the enclosure.

FIG. 12 is a top back perspective view of the top portion, showing ribs on the outside of the top portion, according to another embodiment.

FIG. 13 is a flow chart of the steps for assembling a gas detection sensor arrangement in accordance with an aspect of the present disclosure.

FIGS. 14a through 14e schematically show various configurations for sealing a gas sensor opening. Specifically, FIG. 14a shows a gas sensor opening provided with a flat profile edge; FIG. 14b shows a chambered profile edge; FIG. 14c shows a chamfered parallel profile edge; FIG. 14d shows a rounded profile edge; and FIG. 14e shows a stepped or a seated profile edge.

FIG. 15a schematically shows the electronic assembly, including the PCBA, and an electrical connector low-pressure molded. FIG. 15b schematically shows the electronic assembly, including the PCBA and an electrical cable, low pressure molded. FIG. 15c schematically shows the electronic assembly, including the PCBA and an electrical cable, low pressure molded into the enclosure.

FIG. 16 shows a connector variance and top portion which may be overmolded to thereby provide an integral assembly.

FIG. 17a is a schematic side view of an embodiment of a gas detection sensor arrangement, with the PCBA and the bottom portion removed, showing connector pins insert molded into the top portion, according to a further embodiment. FIG. 17b is a bottom perspective view of a gas detection sensor arrangement according to the embodiment of FIG. 17a.

The scope of the present disclosure is not limited to the above schematic drawings, the number of constituting components, the relative arrangement thereof, etc. These drawings are disclosed simply as examples of embodiments.

### DETAILED DESCRIPTION

With the advent of the use of moderate-to-low GWP refrigerants, such as A2L, the use of refrigerant gas detection sensors for detecting refrigerant gas leaks has become mandatory for indoor units of heating, ventilating, and air-conditioning (HVAC) systems for safety reasons. Further, when using moderate-to-low GWP refrigerants certain safety requirements or regulations must be met. This is especially true when using flammable refrigerants (A3), for example, R290 or R600a. This is also true even when using lower toxic or mildly flammable (A2L) refrigerants, for example, R32 or R1234ze/yf, or blends such as R454B, as such mildly flammable refrigerants have an increased potential to burn as their concentration increases.

Preferably, such refrigerant gas detection sensors are installed within the air handling units of the HVAC systems, e.g., in indoor units of residential HVAC systems. Such units typically include heat exchangers and fans, and leaking of refrigerant is most likely to occur and most critical within these units. Alternatively, the refrigerant gas detection sensors could also be arranged outside the HVAC unit enclosure, for example in air ducts of the HVAC system near the outlet of the unit.

The gas detection sensor arrangement may comprise one or more relays and/or switches communicating with one or more microcontrollers to control one or more auxiliary units, including for example without limitation, a fan, an indicator lamp, an electrically-activated valve solenoids.

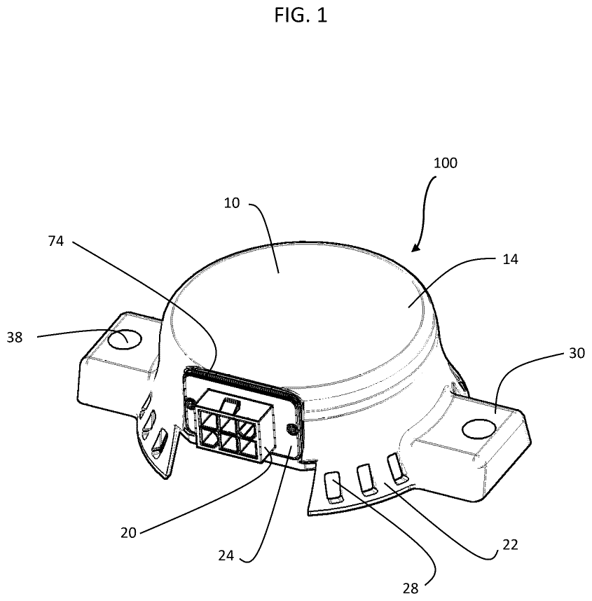

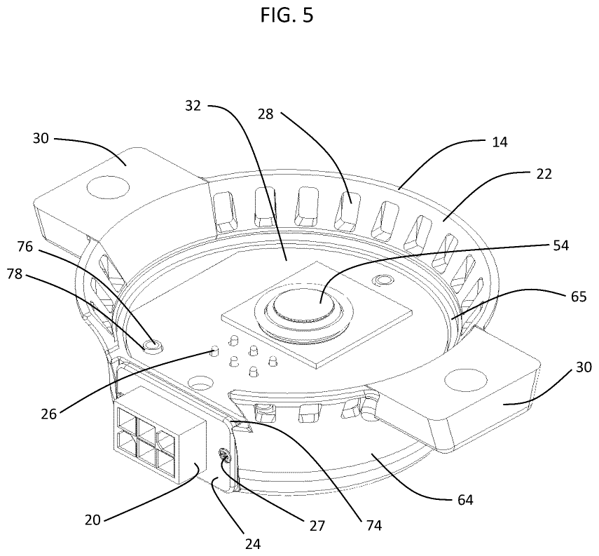

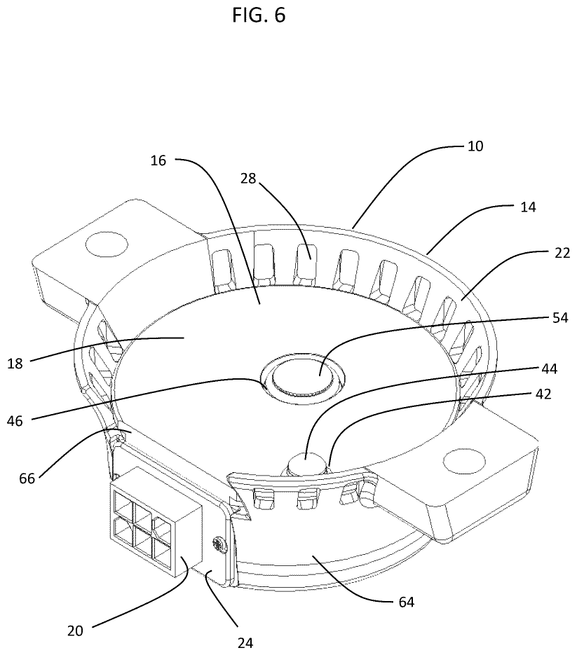

Referring to FIGS. 1 through 6, a gas detection sensor arrangement 100 including an enclosure 10 is shown. The enclosure 10 includes a first portion 14 (e.g., a top portion), a second portion 16 (e.g., a bottom portion) and a connector 20. A sensor 54 configured to detect gas leaks is disposed, at least partially, within the enclosure 10 (see FIGS. 5-6). Further, a circuit board 32, for example, a printed-circuit board assembly (PCBA), is disposed within the enclosure 10.

Materials for forming the various components of the enclosure 10, i.e., the top portion 14, the bottom portion 16 and/or the connector housing, may include a UV-resistant polymer. Further, any of the various components, i.e., the top portion, the bottom portion and the electrical connector housing may be provided with a flame-retardant material. In a preferred embodiment, the flame-retardant material has a minimum UL94 flammability rating of V0.

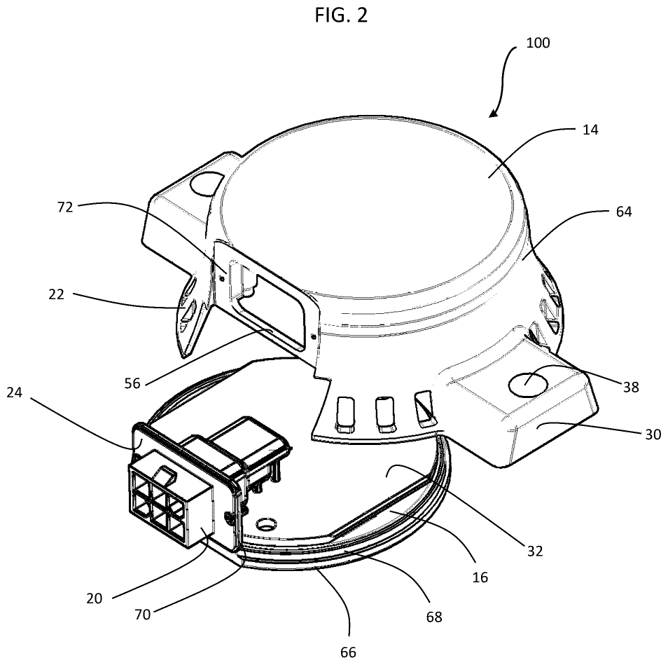

As best shown in FIGS. 2-4, an electrical connector opening 56 is formed in the enclosure 10. In the embodiment of FIGS. 1-6, the electrical connector opening 56 is formed in the top portion 14. The electrical connector opening 56 is configured to receive an electrical connector 20 and includes an electrical connector opening edge 72. The connector 20 includes an adapter flange 24 having an adapter flange edge 70. The electrical connector opening edge 72 is configured to mate with the adapter flange 24.

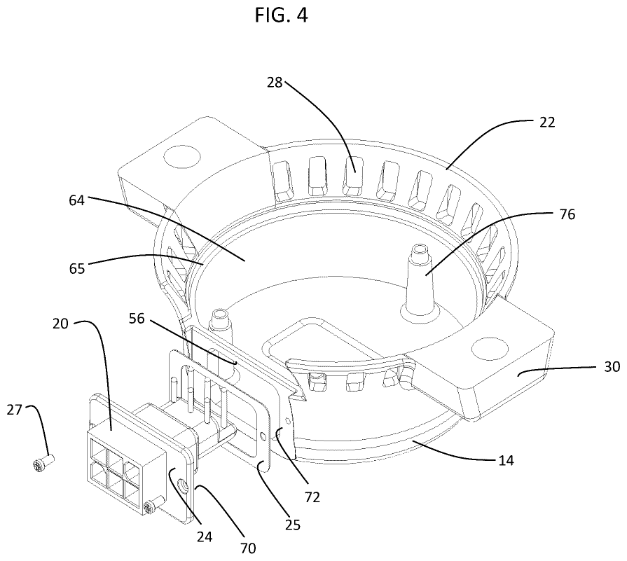

According to this embodiment, as best shown in FIGS. 1 and 4-6, top portion 14 includes a top portion sidewall 64 extending around a perimeter of top portion 14 and also includes a skirted lower portion 22 extending from the top portion sidewall 64. Top portion sidewall 64 is provided with a surface 65 (see FIGS. 4 and 5) configured for connection to bottom portion 16. Surface 65 may be configured as an edge circumferentially extending along an inner surface of the skirted lower portion 22.

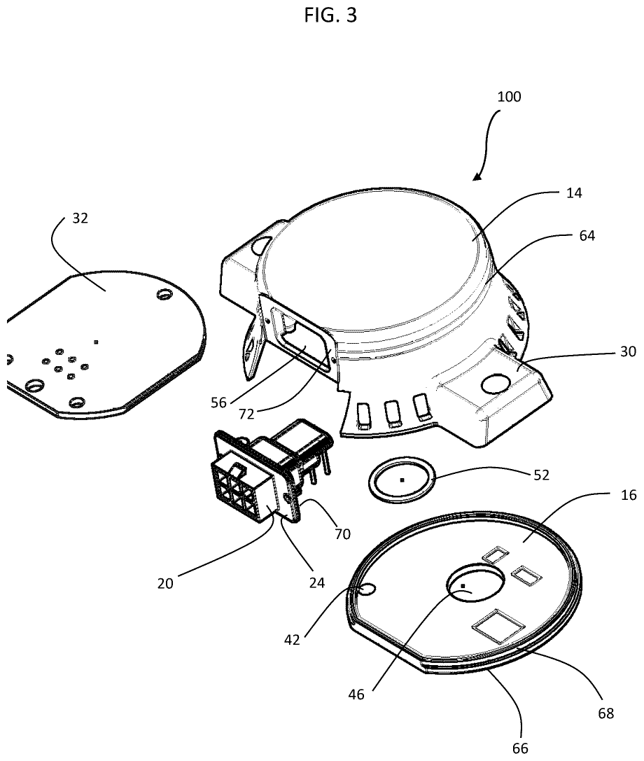

The top portion 14 (or optionally, the bottom portion 16) may include pillars 76 configured to engage with circuit board openings 78 (see, e.g., FIGS. 3 and 5) and secure circuit board 32 to top portion 14 (or bottom portion 16).

As best shown in FIGS. 3 and 6, bottom portion 16 includes a gas sensor opening 46. The gas sensor opening 46 is configured to allow the gas being sensed to reach the gas sensor 54. According to certain embodiments, the gas sensor opening 46 may further be configured to receive at least a portion of the sensor 54. When the gas sensor 54 is mounted within enclosure 10, the perimeter of the gas sensor opening 46 is sealed by the gas sensor 54. Thus, for example, gas sensor 54 may be pressed against the gas sensor opening 46, thereby providing a seal between bottom portion 16 and gas sensor 54. Optionally, gas sensor 54 may be press-fit into the gas sensor opening 46a to form a seal.

In a preferred embodiment, the gas sensor opening 46 may be configured as a through hole in a bottom plate 18 of the bottom portion 16. For example, the gas sensor opening 46 may be configured as a simple through hole in a flat portion of the bottom plate 18 of the bottom portion 16. Thus, gas sensor 54 may be pressed against the flat portion of the bottom plate 18 without any intervening standoff or spacer portion provided around the through hole within the enclosure 10. Further, an optional sealing element 52 may be provided without providing any groove or chamfer in bottom plate 18 of bottom portion 16. In such an embodiment, the sealing element 52 may just lay flat against the bottom plate 18 of bottom portion 16 and be held in place by the gas sensor 54.

Optionally, according to certain embodiments, a groove or chamfer or fillet extending around gas sensor opening 46 and facing the interior of the enclosure 10 may be provided on the bottom plate 18 of bottom portion 16. The groove or chamfer or fillet may be configured to receive the sealing element 52, such as an O-ring, gasket, or other relatively soft sealing material (e.g., UV-cured adhesive or other adhesive), such that when gas sensor 54 is mounted within enclosure 10 and biased toward gas sensor opening 46, a robust seal is formed between bottom portion 16 and gas sensor 54.

According to a preferred embodiment, the top portion 14 and the bottom portion 16 are configured such that, when the top portion 14 and the bottom portion 16 are joined together, thereby creating a sealed enclosure seam 62, the gas sensor 54, sealing element 52 and chamfer extending around the gas sensor opening 46 are aligned and compressed against each other and a seal is formed between gas sensor opening 46 and gas sensor 54.

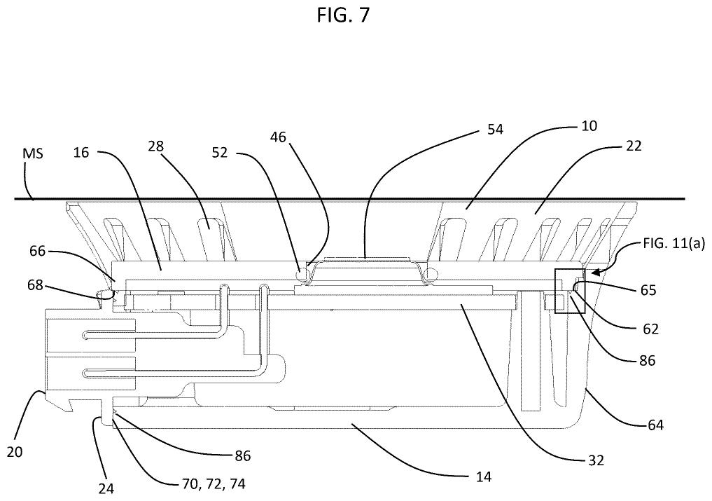

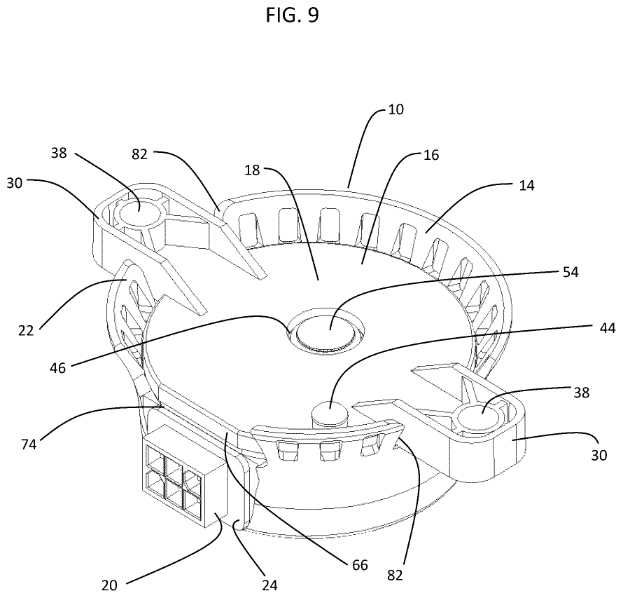

In the embodiment of FIGS. 1-6, the top portion 14 includes mounting legs 30 for attaching the gas detection sensor arrangement 100 to a mounting surface MS (see FIG. 7). The mounting legs 30 may include, for example, mounting through holes 38. Fasteners (not shown) may be inserted into the mounting through holes 38 to secure the mounting legs 30 to a mounting surface. Mounting legs 30 may space an exterior surface of a bottom plate 18 (see FIG. 7) of bottom portion 16 from the mounting surface MS (see FIG. 7). There may be any number of mounting legs 30, typically from one to three. The two mounting through holes 38 as depicted in FIGS. 1-6 are positioned 180° relative to each other, but they can also be oriented at different angles, such as 90°, or both placed next to each other. The mounting legs 30 may be included as part of the top portion 14 or they may be part of the bottom portion 16 as shown in FIG. 9.

Skirted lower portion 22 may extend completely or at least partially (as shown in FIGS. 1-6) along the circumference of top portion sidewall 64. Skirted lower portion 22 may be provided with a plurality of apertures 28. The apertures 28 may be arranged circumferentially around a perimeter of top portion 14. These apertures 28 are provided to facilitate flow of the gas being sensed to the gas sensor 54, while at the same time limiting the exposure of the gas sensor 54 to damaging environmental factors. Thus, apertures 28 in the top portion 14 enable the gas to flow to a gas sensor opening 46, which faces the opposed mounting surface MS, and to thereby be sensed by gas sensor 54. The skirted lower portion 22 around the venting holes also serves the function of protecting the gas sensor 54 if splashing liquid, such as water or oil, hits the enclosure 10.

As best shown in FIGS. 2-4, a bottom portion perimeter 66 is formed around the outer edge of the bottom portion 16. A surface 68 is formed on the edge of the bottom portion perimeter 66. Surface 68 on the bottom portion 16 faces sidewall 64 provided on the top portion 14 and more specifically, faces surface 65 of the top portion sidewall 64. The top portion sidewall 64 and the bottom portion perimeter 66 are configured to attach to each other at surfaces 65, 68 to form enclosure seam 62 (see FIG. 7). More specifically, the surface 65 of the top portion edge 64 and surface 68 of the bottom portion perimeter edge 66 are configured to be joined, thereby forming an enclosure seam 62.

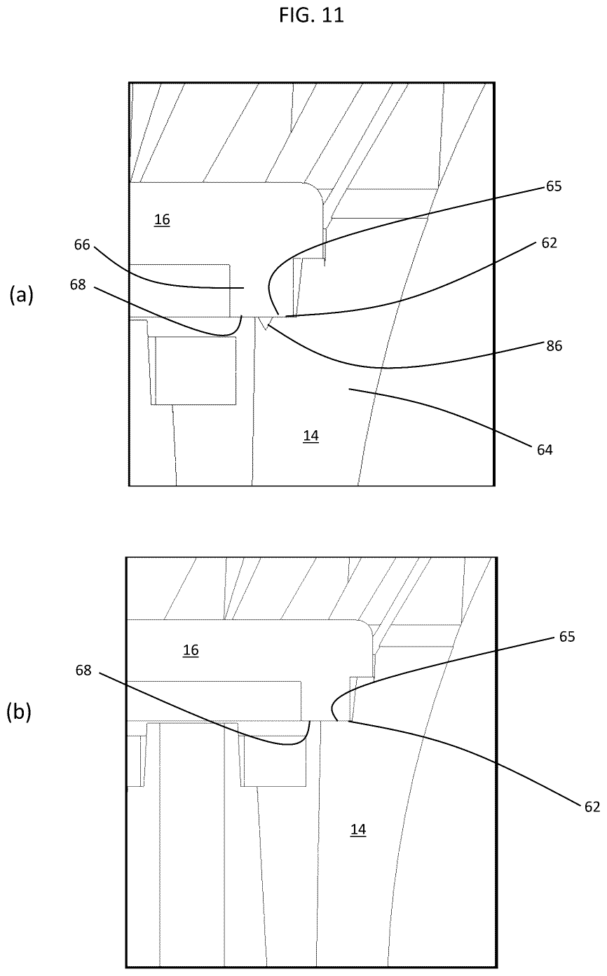

According to preferred embodiments and referring to FIG. 7 and FIG. 11(a) and 11(b), the surface 65 of top portion edge 64 and the opposing surface 68 of bottom portion sidewall 66 are connected through welding, particularly ultrasonic welding or laser welding, to thereby form the enclosure seam 62. Welding of the enclosure seam 62 seals the enclosure 10, resulting in a mechanically robust connection that is advantageously resistant to environmental conditions, such as frost, and pressurized water jets used to clean the enclosure 10.

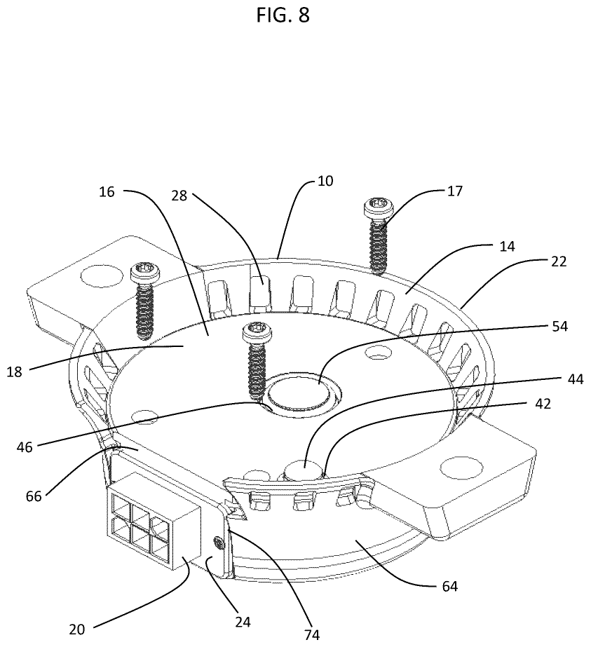

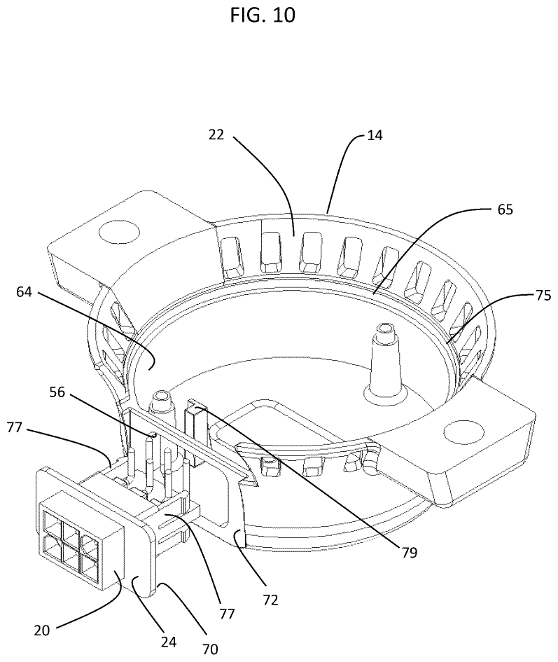

In another embodiment, as best shown in FIGS. 8 and 10, the surface 65 of top portion edge 64 and the opposing surface 68 of bottom portion sidewall 66 may be joined using fasteners 17 (see FIG. 8) to thereby form the enclosure seam 62. Any number of fasteners 17 may be used. Further, a sealing element 75, as best shown in FIG. 10, may extend around the perimeter of the 65, 65 between the opposing surfaces of the top portion 14 and the bottom portion 16. Sealing element 75 may ensure that the top portion 14 and the bottom portion 16 are sealed when the fasteners 17, e.g., screws, are tightened. Sealing element 25 may be provided as a separate element such as an O-ring, gasket, or as a relatively soft polymer co-molded to at least one of the opposing surfaces, or as other sealing material, including UV-cured adhesives or epoxy, or potted using a silicone filler, such as a one-or two-part silicone.

As shown in the alternative embodiment of FIG. 9, bottom portion 16 of the enclosure 10 may include a bottom plate 18 surrounded by the bottom portion sidewall 66, mounting legs 30 each with a mounting through hole 38, a gas sensor opening 46, and a light guide through hole 42. The bottom portion sidewall 66 extends from the bottom plate 18 and projects in a direction towards sidewall 64 of the top portion 14. Bottom portion sidewall 66 further includes a surface 68 that faces opposes a surface 65 of top portion 14. As discussed below and as shown in FIGS. 7, 11(a), 13(a) and 13(b), an energy director 86 may be provided on surface 68 of bottom portion sidewall 66 (or on surface 65 of top portion sidewall 64) to facilitate ultrasonic welding of the bottom portion 16 to the top portion 14.

According to other embodiments, the top portion 14 and the bottom portion 14 may be joined and sealed by snap-fit features similar to the snap-fit features 77, 79 used to join connector 20 to enclosure 10 (see FIG. 10 and associated disclosure, below). According to even other embodiments, the opposing surfaces 65, 68 may be joined and sealed by adhesive bonding (e.g., via UV curing adhesives or epoxy, or potted using a silicone filler, such as a one- or two-part silicone.)

As is known in the art, one or more electronic components (which may include microcontrollers, relays, switches, etc.) are disposed on one or both sides of the circuit board 32. In certain embodiments, the electronic components may comprise one or more relays and/or switches to control one or more auxiliary units associated with the operation of the HVAC system, including for example without limitation, a fan, an indicator lamp, an electrically-activated valve solenoid, and/or other components that could mitigate the effects of a detected gas leak and enhance the safety of the system.

In a preferred embodiment, the electronic components (other than the gas sensor 54 and optionally a gas sensor PCBA) are disposed on an upper side of the circuit board 32. The gas sensor 54 (see FIGS. 5-7) and optionally the gas sensor PCBA may be disposed on a lower surface of the circuit board 32, i.e., a surface facing the gas sensor opening 46 and the mounting surface MS. Circuit board 32, with components on one or both sides, may be soldered to the integrated pins of the various connectors 20 or otherwise mounted to the enclosure 10, for example, using surface-mounted sockets or press fit pins.

Referring to FIGS. 1-6, the enclosure 10 seals and protects the circuit board 32, the electronic components mounted on the board 32 (including, for example, a sensor PCBA), and the gas sensor 54 from the potentially damaging environment external to enclosure 10. In the embodiment of FIGS. 1-6, the top portion 14 is configured to accommodate the electronic components located on the upper side of circuit board 32. The bottom portion 16 is configured to accommodate the gas sensor 54 and/or the sensor PCBA located on the lower side of circuit board 32. Thus, for example, the bottom portion sidewall 66 may be configured to provide sufficient space for the gas sensor 54 to be disposed between the underside of circuit board 32 and gas sensor opening 46. In an assembled state, circuit board 32, with gas sensor 54 located on the underside of circuit board 32, may be disposed on pillars 76 within top portion 14 (see FIG. 4).

When, enclosure seam 62 is formed, gas sensor 54 is pressed against the perimeter surface of gas sensor opening 46 and/or against sealing element 52 which is correspondingly is pressed against the perimeter surface of the gas sensor opening 46 in the bottom plate 18 of bottom portion 16. Gas sensor 54 and/or sealing element 52 thus secures the gas sensor 54 in place across the gas sensor opening 46, thereby allowing the sensor 54 to perform its operational purpose. In this secured position, the gas sensor 54 is configured to detect gas coming through the aperture holes 28 of enclosure 10. As discussed above, according to some embodiments, the gas sensor opening 46 and/or the sealing element 52 are configured to accommodate the outer perimeter of the gas sensor 54.

Still referring to FIGS. 1-6, in a preferred embodiment, opposing edges of a side wall of top portion 14 and bottom perimeter wall 66 of bottom portion 16 are connected at an enclosure seam 62. Preferably, enclosure seam 62 is sealed by ultrasonic welding.

In FIGS. 1-6, connector 20 is shown as a six-pin connector. In general, the assembled enclosure 10 may utilize any of various different connectors. For example, connector 20 may be any connector suitable for providing input/output capability to the circuit board (e.g., a five-pin connector, a six-pin connector, an eight-pin connector, etc.).

Still referring to FIGS. 1-6 an adapter flange 24 is formed around the connector 20. An adapter flange edge 70 (see FIGS. 2-5) is formed around the perimeter of the adapter flange 24. The adapter flange edge 70 and the electrical connector opening edge 72 are configured to connect, forming a connector adapter flange seam 74 (see FIGS. 1 and 5). The adapter flange 24 and adapter flange edge 70 of these various connectors 20. may have a standard or common configuration so that all the connectors 20 may engage with an electrical connector opening edge 72 having a standard or common configuration. The ability to easily use different connectors 20 increases the versatility of the enclosure 10 in enabling different power sources to be connected to the pins 26 of the arrangement 100.

As shown in FIGS. 1-6, the adapter flange edge 70 and the electrical connector opening edge 72 may be connected at the connector adapter flange seam 74 by fasteners 27. A sealing element 25, as best shown in FIG. 4, may extend around the perimeter of the adapter flange 24 between the opposing surfaces of the adapter flange edge 70 and the electrical connector opening edge 72. Sealing element 25 may ensure that the adapter flange seam 74 is sealed when the fasteners 27, e.g., screws, are tightened.

Sealing element 25 may be provided as an O-ring, gasket or relatively soft polymer or other sealing material, including UV-cured adhesives. For example, a soft polymer may be co-molded on the connector opening edge 72 or co-molded on the adapter flange edge 70. To tightly secure the connector 20 to the enclosure 10 one or more fastening elements may be used, for example, screws 27 as shown in FIGS. 1-6.

Referring back to FIG. 3 and also to FIGS. 6, 8 and 9, a light guide 44 may extend through a light guide through hole 42 provided in the bottom portion 16. In a preferred embodiment, the light guide through hole 42 is located on bottom plate 18. The light guide 44 is a device used to direct light from a light source, e.g., an LED, to a place where the light may be visible. According to a preferred embodiment, the light from the light guide 44 may be projected onto a surface on which the gas detector sensor arrangement 100 is mounted, with the light then being reflected out through the aperture holes 28. The light source and the light guide 44 may assist in locating the gas detection sensor arrangement 100 when the enclosure 10 is not otherwise clearly visible. Optionally, the light source and the light guide 44 may be used to indicate the operational status of the gas detection sensor arrangement, for example, with blinking or colored-coded lights.

The light guide 44 may be added as a separate component into the bottom portion 16 using a press fit mounting mechanism, fasteners, snap fittings or welding. Optionally, the light guide 44 may be replaced by a bottom portion 16 having at least a section made of a clear, transparent polymer or a translucent polymer which can transmit light from the light source.

In an alternative embodiment, as shown in FIG. 9, the mounting legs 30 may be attached to the bottom portion 16. In this alternative embodiment, the skirted lower portion 22 of top portion 14 may include slots, openings or gaps 82 in the skirted lower portion 22 configured to receive the mounting legs 30. These slots, openings or gaps 82 allow the mounting legs 30 to extend beyond the circumferential, thin-walled skirted lower portion 22 of the top portion 14.

Also as shown in FIG. 9, the adapter flange edge 70 of connector 20 and the electrical connector opening edge 72 of enclosure 10 may be connected at the connector adapter flange seam 74 by welding, for example, ultrasonic welding (see FIG. 7) or laser welding, etc. Ultrasonically welding the flange 24 to the rest of the enclosure 10 may provide a cost-effective assembly, as the same ultrasonic horn can be used to ultrasonically weld the various different connectors 20 to the electrical connector opening 56 when the various connectors 20 are provided with a standard or common configuration for the adapter flanges 24. According to other embodiments, the opposing surfaces 70, 72 may be joined and sealed by adhesive bonding (e.g., via epoxy or potted using a silicone filler, such as, a one-or two-part silicone).

In other embodiments, as shown in FIG. 10, the adapter flange edge 70 and the electrical connector opening edge 72 may be connected at the connector adapter flange seam 74 by the engagement of one or more connector snap fittings 77 with one or more top portion snap fittings 79 associated with either the top portion 14 (as shown in FIG. 10) or the lower portion 16 (not shown). For example, to join the connector 20 to the enclosure 10, a hook of a snap lock fitting 77 provided on the connector 20 may elastically flex upon initial contact with a flange of a snap-fitting tower 79 integral provided with the top portion 16. The hook of the snap lock 77 may then slide past the flange of the tower 79 and subsequently elastically snap back to its unflexed position, thereby allowing the hook of the snap lock 77 to engage the flange of the tower 79 when the opposing surface 70, 72 (with any optional sealing element 25 therebetween) are engaged. Additionally, such snap-fit features may be used to secure and hold the connector 20 to the enclosure 10 prior to welding (ultrasonic or laser) or during adhesive bonding surfaces 70, 72 together.

Referring now to FIGS. 6 and 7 and also to FIG. 11(a), the bottom portion 16 is shown ultrasonically welded to the top portion 14. An energy director 86 may be used to facilitate the joining of the two portions. In FIG. 11(a), the energy director 86 is provided on sealing surface 68 of bottom portion 16. Specifically, energy director 86 is provided on the sealing surface 68 of the sidewall 66 of bottom portion 16. Sealing surface 68 opposes the sealing surface 65 that is provided on a sidewall 64 of top portion 14. Alternatively, the energy director 86 may be provided on top portion 14. Specifically, energy director 86 may be provided on the sealing surface 65 of the sidewall 64 of top portion 14. As a further alternative, an energy director 86 may be provided on sealing surface 65 of top portion 14 and an energy director 86 may be provided on sealing surface 68 of bottom portion 16. During ultrasonic welding of surface 65 to surface 68, the energy director 86 acts as a point of high stress that softens and melts under the application of energy. This facilitates the welding of surface 65 to surface 68 by focusing and directing the ultrasonic energy where the energy directors 86 are located.

FIG. 11(a) shows the enclosure seam 62 formed after surface 65 has been ultrasonically welded to surface 68. Energy director 86 has melted, at least partially, and surfaces 65 and 68 have been brought together to form the enclosure seam 62. In a preferred embodiment, energy director 86 is formed of the same material as sidewall 64 (or sidewall 66) upon which energy director is located. Alternatively, the energy director and the sidewall 64 or sidewall 66 are formed of different materials. Energy director 86 is a protrusion extending above surface 65, 68 that may be integrally formed with sidewall 64, 66 or may be subsequently located on surface 65, 68 after the initial formation of sidewall 64, 66. In one embodiment, energy director 86 may be formed as a ridge that continuously extends around the entire perimeter formed by sidewalls 64, 66. In another embodiment, energy director 86 may be formed as a series of ridges, with gaps formed therebetween, or even as a series of beads, as it extends around the perimeter formed by sidewalls 64, 66. Further, energy director 86 need not be formed with a triangular cross-section, but may be formed with a pyramidal cross-section or even with a mounded or rounded cross-section, or any other form that allows the energy to be focused and to thereby promote melting.

Similarly, referring now to FIG. 7, an energy director 86 may be provided on electrical connector 20 to facilitate the ultrasonic welding of connector 20 to enclosure 10. Specifically, in FIG. 7, energy director 86 is provided on the adapter flange edge surface 70 of the adapter flange 24 of electrical connector 20. Referring now also to FIG. 1, adapter flange edge surface 70 opposes the electrical connector opening edge surface 72 that is provided at the electrical connector opening 56 formed in top portion 14. The opposing surfaces of the adapter flange edge surface 70 and the electrical connector opening edge surface 72 may be configured to be connected by ultrasonic welding to form a connector adapter flange seam 74. Alternatively (not shown), energy director 86 may be provided the enclosure 10, for example, on electrical connector opening edge surface 72 of top portion 14. As a further alternative, an energy director 86 may be provided on electrical connector opening edge surface 72 of top portion 14 and an energy director 86 may be provided on adapter flange edge surface 70 of electrical connector 20. During ultrasonic welding of surface 70 to surface 72, the energy director 86 acts as a point of high stress that softens and/or melts first under the application of ultrasonic energy. This facilitates the welding of surface 70 to surface 72 by focusing and directing the ultrasonic energy where the energy directors 86 are located.

Referring now to the embodiment shown in FIG. 11(b), the bottom portion 16 is shown laser welded to the top portion 14. For laser welding, a shear joint formed with two opposing complementary surfaces, may be used. In FIG. 11(b), surface 68 of the bottom portion 16 and surface 65 of the top portion 14 are shown as two flat opposing surfaces. A welding laser beam is directed at the interface of the surfaces 65, 68 such that the energy from the laser beam is absorbed by one or more of the surfaces 65, 68. The energy causes one or more of the surfaces 65, 68 to melt, thereby subsequently joining the surfaces 65, 68 when the energy is removed. In a preferred embodiment, the material of one of the bottom portion 16 or the top portion 14 can transmit the wavelength of the welding laser beam, thereby allowing the energy from the laser beam to be transmitted by the first portion and to be absorbed by the surface of the other portion. For example, the material of the bottom portion 16 may transmit the wavelength of a welding laser beam that is directed at the interface of the surfaces 65, 68 such that the energy from the laser beam is absorbed by the surface 65 of the top portion 14. When the energy is absorbed, by surface 65, surface 65 melts, and enclosure seam 62 is formed when the mating surfaces cool.

Similar to the formation of the enclosure seam 62 using laser welding, the connector seam 74 may be formed by laser welding the connector 20 to the enclosure 10. For example, the adapter flange edge surface 70 and the electrical connector opening edge surface 72 may be provided as two opposing complementary surfaces, for example, as two flat opposing surfaces. A welding laser beam is directed at the interface of the surfaces 70, 72 such that the energy from the laser beam is absorbed by one or more of the surfaces 70, 72. The energy causes one or more of the surfaces 70, 72 to melt, thereby subsequently joining the surfaces 70, 72 when the energy is removed. In a preferred embodiment, the material of one of the connector adapter flange 24 or the electrical connector opening 56 can transmit the wavelength of the welding laser beam, thereby allowing the energy from the laser beam to be transmitted by the flange 24 (or the opening 56) and to be absorbed by the surface of the other portion. For example, the material of the connector adapter flange 24 may transmit the wavelength of a welding laser beam that is directed at the interface of the surfaces 70, 72 such that the energy from the laser beam is absorbed by the surface 72 of the electrical connector opening 56. When the energy is absorbed, by surface 72, surface 72 melts, and connector seam 74 is formed when the mating surfaces cool.

As disclosed herein, according to certain aspects, a gas detection sensor arrangement 100 may include an enclosure 10 having a first portion 14 and a second portion 16 (for example, a top portion and a bottom portion), a circuit board 32 disposed within the enclosure 10, and a gas sensor 54 for detecting gas that is present in an environment external to the enclosure 10. The gas sensor 54 is disposed at least partially within the enclosure 10. The first portion 14 and the second portion 16 are brought together to seal the circuit board 32 within the enclosure 10.

The enclosure 10 may include an electrical connector opening 56 and an electrical connector 20. The electrical connector 20 has an electrical connector adapter flange. The electrical connector opening 56 and the electrical connector adapter flange 24 have opposing surfaces (for example, surfaces 70 and 72) configured to be brought together to seal the electrical connector opening 56.

The enclosure 10 includes a gas sensor opening 46, which is configured to allow gas in an environment external to the enclosure 10 to reach the gas sensor 54. When the first portion 14 and the second portion 16 are brought together the gas sensor 54 seals the gas sensor opening 46. In a preferred embodiment, the enclosure 10 may act as a clamp that biases the gas sensor 54 towards the gas sensor opening 46 to thereby seal the gas sensor opening 46.

According to certain embodiments, one more microcontrollers, relays, switches, or other electronic components may be disposed on a first side of the circuit board 32. The first side of the circuit board 32 may be accommodated within the first portion 14 of the enclosure 10 and may face away from the gas sensor opening 46. The gas sensor opening 46 may be provided in the second portion 16 of the enclosure 10. Thus, the electronic components that are located on the first side of the circuit board 32, which faces away from the gas sensor opening 46, are further protected should a leak develop at the gas sensor opening 46.

Additionally, a sealing member 52, such as an O-ring, a gasket or a relatively soft polymer, may be provided adjacent the gas sensor opening 46. This sealing member 52 may be compressed between the gas sensor 54 when the first portion 14 and the second portion 16 are joined (e.g., by welding, mechanically fastening, adhesive bonding, snap fitting, etc.). Thus, the sealing member 52 may assist in sealing the gas sensor opening 46.

According to another embodiment, a light guide opening 42 may be provided in the enclosure 10, preferably in the second portion 16. A light guide 44 may be inserted into the light guide opening 42 to thereby seal the light guide opening 42. Optionally, at least a portion of the second portion 16 (or at least a portion of the first portion 14) may be transparent or translucent, such that light from a light source, e.g., an LED, within the enclosure 10 may be externally visible.

According to other aspects, components for an enclosure 10 for a gas detection sensor arrangement 100 may be provided. The components include a first component 14 of the enclosure 10 and a second component 16. At least one of the first component 14 or the second component 16 is configured to accommodate a circuit board 32. At least one of the first component 14 or the second component 16 is configured to accommodate a gas sensor 54 for detecting a gas in an environment external to the enclosure 10. The first component 14 and the second component 16 are configured to be joined together to form the enclosure 10 around the circuit board 32. In a preferred embodiment, the first component 14 and the second component 16 are configured to be joined together to form a sealed enclosure 10. In a further preferred embodiment, the first component 14 and the second component 16 are configured to be joined together via welding, for example, ultrasonic welding or laser welding.

According to even other aspects, the first component 14 and the second component 16 may optionally be configured to be joined together via mechanical fasteners, snap-fit features or adhesive bonding. Thus, for example, the joining of the first component 14 and the second component 16 into an enclosure 10 may be performed via a plurality of complementary snap-fit features (similar to the snap-fit features 77, 79 associated with joining the connector 20 to the enclosure 10). For example, complementary cantilever or annular snap-fit elements may be molded around the perimeter edges of the first component 14 and the second component 16. Elastic deformation of the snap-fit elements allows them to slide past each other and engage each other to thereby locks the components 14, 16 together. It is expected that the snap-fit features associated with the first and second components 14, 16 will vary in dimension and curvature e.g., annular or cantilever snap fits, from the snap-fit features 77, 79 associated with the connector 20.

When mechanical fasteners or snap-fit features are used to join the first component 14 and the second component 16 a sealing element 75 may additionally be provided between surfaces 65, 68 to facilitate sealing the enclosure 10. Sealing element 75 may be provided as an O-ring, gasket or relatively soft polymer or other sealing material. For example, a soft polymer may be installed as a separate component during assembly, or may be co-molded on surface 65 or co-molded on surface 68. Optionally, the snap-fit features may be used to secure and hold first component 14 and second component in place during a subsequent welding (ultrasonic or laser) operation or during a subsequent adhesive bonding operation.

According to certain embodiments, the components may further include an electrical connector 20 having an electrical connector adapter flange 24. At least one of the first component 14 or the second component 16 includes an electrical connector opening 56 configured to accommodate the electrical connector 20. Opposing surfaces of the electrical connector adapter flange 24 and the electrical connector opening 56 are configured to be joined together to seal the electrical connector opening 56. In a preferred embodiment, the electrical connector adapter flange 24 and the electrical connector opening 56 are configured to be joined by welding, preferably ultrasonic welding or laser welding, to seal the electrical connector opening 56.

According to other embodiments, at least one of the opposing surfaces of the electrical connector adapter flange 24 and the electrical connector opening 56 has an energy director 86 located thereon. The energy director 86 is to be positioned between the opposing surfaces when the electrical connector adapter flange 24 and the electrical connector opening 56 are being joined together. The energy director 86 is configured to initially space the opposing surfaces of the electrical connector adapter flange 24 and the electrical connector opening 56 apart. The energy director 86 is further configured to soften and to, at least partially, melt under an application of ultrasonic energy to the opposing surfaces such that the opposing surfaces of the electrical connector adapter flange 24 and the electrical connector opening 56 are subsequently brought together and ultrasonically welded together during the joining process, thereby sealing the electrical connector opening 56

According to another embodiment, at least one of the first component 14 or the second component 16 includes a gas sensor opening 46 configured to allow gas that is present in an environment external to the enclosure 10 to reach the gas sensor 54. The gas sensor opening 56 is configured to be sealed by the gas sensor 54. Optionally, a sealing member 52, such as an O-ring, gasket, relatively soft polymer, adhesive bond, etc., is disposed between the gas sensor 54 and the at least one of the first component 14 or the second component 16 in which the gas sensor opening 46 is formed. Sealing member 52 may further assist in sealing the gas sensor opening 46.

According to another embodiment, opposing surfaces 65, 68 of the first component 14 and the second component 16 are configured to be joined, preferably welded, e.g., laser welded, and more preferably ultrasonic welded, together at an enclosure seam 62. The opposing surfaces need not be flat, but may include steps, channels, protrusions, or in general, any complementary surface geometries. At least one opposing surface of the first component 14 or the second component 16 may have an energy director 86 located thereon. The energy director 86 is configured to initially space the opposing surfaces of the first component 14 and the second component 16 apart during the formation of the enclosure seam 62. The energy director 86 is further configured to focus heat generation at the interface of the opposing surfaces. Thus, the energy director 86 may soften and/or melt sooner than the opposing surfaces under an application of energy to the enclosure seam 62. As the energy director 86 softens/melts, the opposing surfaces of the first component 14 and the second component 16 are brought together and ultrasonic welded together thereby forming the enclosure seam 62 and sealing the enclosure 10.

According to a further embodiment, a gas sensor opening 46, which is configured to allow gas that is present in an environment external to the enclosure 10 to reach the gas sensor 54, is provided in the one of the first component 14 or the second component 16. The gas sensor opening is configured to accommodate the gas sensor 54 or at least a portion thereof.

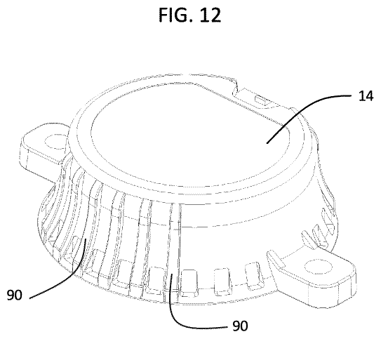

Referring now to FIG. 12, the top portion 14 may include a plural number of ribs 90 preferable oriented in the vertical direction and spaced around at least a portion of the outer or inner surface. Ribs 90 stiffen the top portion 14, which may be advantageous during assembly. Further, the ribs 90 increase the strength and impact resistance of the top portion 14 which may be advantageous if the enclosure should be dropped, e.g., to the floor, thus preventing the top portion from cracking or breaking. The bottom portion 16 may also include ribs.

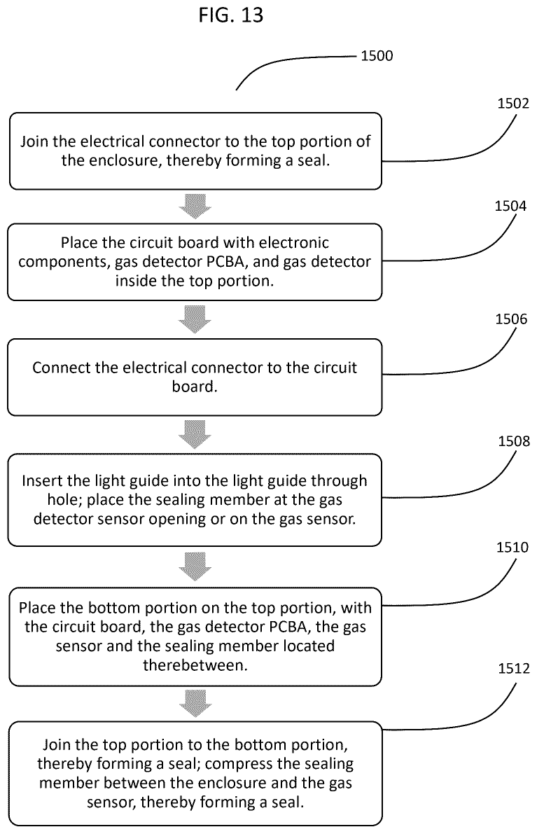

Referring to FIG. 13, a method 1500 for assembling the gas detection sensor arrangement 100 is provided. At step 1502, opposing surfaces of the electrical connector 20 and the top portion 14 of the enclosure 10 are brought together and joined to form a sealed seam. More specifically, opposing surfaces of the electrical connector adapter flange 24 and the electrical connector opening 56 may be joined together, preferable by ultrasonically welding. At step 1504, the circuit board 32, the electronic components 36, the sensor PCBA 34, and the sensor 54 are placed inside the top portion 14. For example, circuit board 32 may be mounted to pillars 76 associated with top portion 14, by inserting the pillars 76 into circuit board openings 78. At step 1506, the circuit board 32 is connected to the pins or terminals 26 of electrical connector 20. At step 1508, the light guide 44 may be inserted into the light guide through hole 42 and the sealing member 52 may be sub-assembled to either the gas detector sensor opening 46 or to the gas sensor 54, itself. Note that step 1508 may take place before or after any of steps 1502, 1504, 1504 or 1506. At step 1510, the bottom portion 16 is placed on the top portion 14 with the circuit board 32, the sensor PCBA 34, the sensor 54, and an optional sealing member 52 disposed therein. At step 1512, opposing surfaces of the bottom portion 16 and of the top portion 14 are joined together, preferable by ultrasonically welding, to form a seal. In the process of joining the bottom portion 16 to the top portion 14, the sealing member 52 may be compressed between the gas sensor 54 and the enclosure 10 at the gas detector sensor opening 46.

According to an embodiment, an opposing surface of the bottom portion 16 may be placed on the opposing surface of the top portion 14, with the circuit board 32, the gas sensor 54 and the sealing member 52 located within the volume between the bottom portion 16 and the top portion 14. Notably, an energy director 86, which, for example, may be located on the opposing surface of bottom portion 16, spaces the opposing surface of the bottom portion 16 a predetermined distance away from the opposing surface of the top portion 14. The predetermined distance is, at least nominally, the height of the energy director 86 projecting from the opposing surface. At this stage in the process (after step 1510 and before step 1512), a sealing member 52, which is located between the gas sensor 54 and an interior surface of the bottom portion 16 at the gas detector sensor opening 46, is not compressed (or only lightly compressed so as to maintain its alignment).

After step 1512, the energy director 86, which was located on the opposing surface of bottom portion 16 has melted and now forms part of the enclosure seam 62 between the bottom portion 16 and the top portion 14. Specifically, as the ultrasonic energy for joining the bottom portion 16 and the top portion 14 was supplied, the energy director 86 melted. As the energy director 86 melted, the opposing surfaces were brought together until they finally contacted each other to form a sealed seam 62. In the process, the sealing member 52, which is located between the gas sensor 54 and an interior surface of the bottom portion 16 at the gas detector sensor opening 46, becomes further compressed, thereby providing a robust seal between the enclosure 10 and the gas sensor 54. In the gas detection sensor arrangement 100, the enclosure 10 essentially acts as a clamp that secures and compresses the sealing member 52 between the gas detector sensor opening 46 and the gas sensor 54.

Thus, as presented above, according to an additional aspect, a method for assembling a gas detection sensor arrangement 100 is provided. The method includes providing a first portion 14 of an enclosure 10 for the gas detection sensor arrangement 100 and providing a second portion 16 of the enclosure 10 for the gas detection sensor arrangement 100. An electrical connector 20 having an electrical connector adapter flange 24 is provided. The electrical connector adapter flange 24 is joined (using, for example, welding, e.g., ultrasonically welding or laser welding, mechanically fasteners, snap-fit features or adhesive bonding) to an electrical connector opening 56 provided in one of the first portion 14 or the second portion 16. A circuit board 32 having a gas sensor 54 disposed thereon is operatively assembled to pins 26 of the electrical connector 20. The first portion 14 is joined (using, for example, welding, e.g., ultrasonically welding or laser welding, mechanically fasteners, snap-fit features or adhesive bonding) to the second portion 16 at an enclosure seam 62 to seal the circuit board 32, and all the other electronic components, inside the enclosure 10.

Thus, according to one embodiment, the method for assembling a gas detection sensor arrangement 100 may further include placing a sealing member 52 between the gas sensor 54 and one of the first portion 14 or the second portion 16 and compressing the sealing member 52 between the gas sensor 54 and the one of the first portion 14 or the second portion 16 during the step of joining the first portion 14 to the second portion 16 (see, e.g., FIG. 7).

According to certain embodiments, a step of ultrasonically welding the electrical connector adapter flange 24 to the electrical connector opening 56 preferably includes: providing an energy director 86 between opposing surfaces 70, 72 of the electrical connector adapter flange 24 and the electrical connector opening 56; softening and then, at least partially, melting the energy director 86; and then bringing the opposing surfaces 70, 72 of the electrical connector adapter flange 24 and the electrical connector opening 56 together as the energy director 86 melts.

Similarly, according to certain embodiments, the step of ultrasonically welding the first portion 14 to the second portion 16 includes: providing an energy director 86 between opposing surfaces 65, 68 of the first portion 14 and the second portion 16: softening and then, at least partially, melting the energy director 86: and then bringing the opposing surfaces 65, 68 of the first portion 14 and the second portion 16 together at the enclosure seam 62 as the energy director 86 melts.

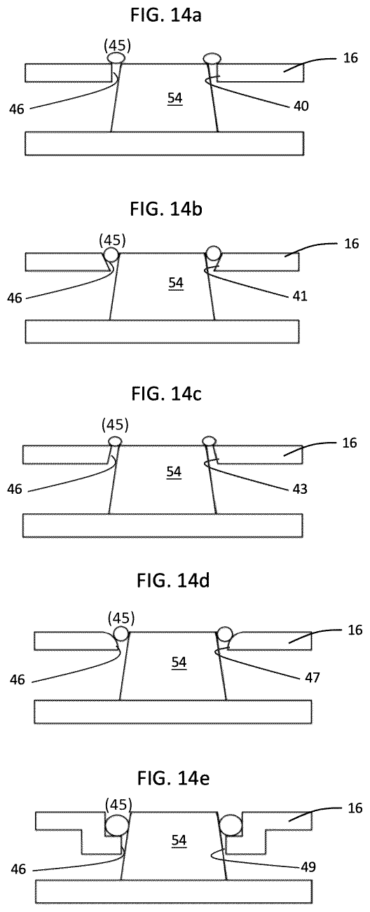

In alternative embodiments, referring to FIGS. 14a through 14e, to ease the assembly process and to seal the gap between the gas sensor opening 46 (or the second portion 16) of the housing 10 and the gas sensor 54, the sealing member 52 may be located on an external surface of the housing 10. Specifically, the sealing member 52 may be provided where the circumferential edge of the gas sensor 54 extends adjacent to the gas sensor opening 46. In such case, in a preferred embodiment, sealing member 52 may be provided as a sealant 45, for example, a UV-curing adhesive.

Thus, according to other embodiments, FIGS. 14a through 14e schematically show various configurations for sealing the gas sensor opening 46. As shown in FIGS. 14a through 14e, various options for shaping or contouring the profile edge of the gas sensor opening 46 which faces the circumference of the gas sensor 54 may be provided. Specifically, FIG. 14a shows bottom portion 16 provided with a flat edge contour 40 at gas sensor opening 46 and sealed with sealant 45. FIG. 14b shows bottom portion 16 provided with a chamfered edge contour 41 at gas sensor opening 46 and sealed with sealant 45. FIG. 14c shows bottom portion 16 provided with a chamfered parallel edge contour 43, i.e., a chamfered profile edge that is parallel to the side wall of the gas sensor 54, at gas sensor opening 46 and sealed with sealant 45. FIG. 14d shows bottom portion 16 provided with a rounded edge contour 47 at gas sensor opening 46 and sealed with sealant 45. FIG. 14e shows bottom portion 16 provided with a stepped or a seated edge contour 49 at gas sensor opening 46 and sealed with sealant 45. In all of these embodiments, the sealing member 52 extends around the gas sensor opening 46 formed in the one of the first portion 14 or the second portion 16 and contacts gas sensor 54, thereby sealing this opening.

According to even other embodiments, gas sensor opening 46 may be provided as a press-fit configured to accommodate the circumference of gas sensor 54 and to seal the enclosure 10. Optionally, in addition to press-fitting gas sensor 54 within gas sensor opening 46, a sealing member 52 (such as sealant 45) may be provided.

Electronic components, including PCB assembly 32 are disposed within the housing. In a preferred embodiment, the electronic components 29 (other than the gas sensor 54 and optionally a gas sensor PCBA) may be disposed on an upper side of the circuit board 32. The gas sensor 54 (see FIGS. 5-7) and optionally the gas sensor PCBA may be disposed on a lower surface of the circuit board 32, i.e., a surface facing the gas sensor opening 46 and the mounting surface MS. Circuit board 32, with components on one or both sides, may be soldered to the integrated pins of the various connectors 20 or otherwise mounted to the enclosure 10, for example, using surface-mounted sockets or press fit pins.

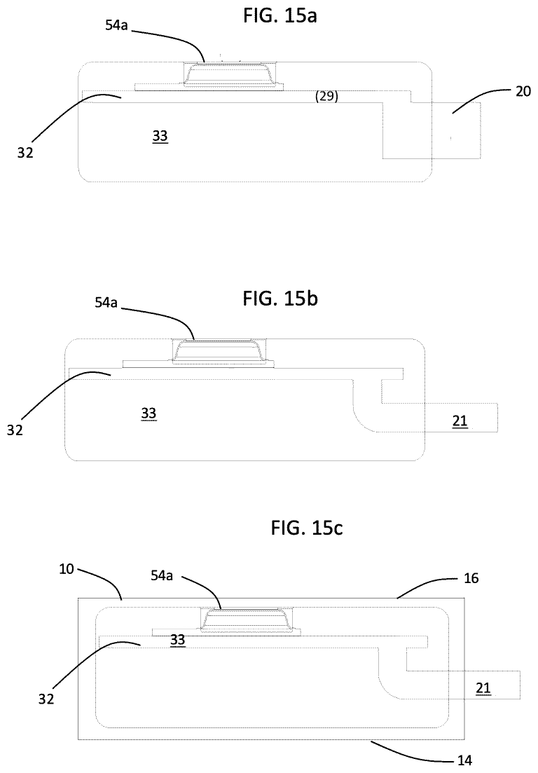

According to certain aspects, as shown in FIGS. 15a and 15b, the electronic components 29 may be encapsulated using low-pressure injection molding. This method provides partial, and preferably complete, sealing of the electronic components 29 including PCB assembly 32, and at least partial sealing of electrical connectors 20 and cable assemblies 21 (i.e., those portions located with the housing 10). The surface 54a of gas sensor 54 is excluded from the molding process. The low-pressure molding encapsulation 33 of the electronics assembly 29 may be used as the final housing and may include mounting features formed by the molding. Alternatively, as shown in FIG. 15c, the low-pressure molding encapsulation 33 of the electronics assembly 29 may be mounted into a conventionally injection molded part (e.g., a top portion 14 or bottom portion 16) for added features. Even further the electronic assembly 29 and a conventionally injection molded part (e.g., a top portion 14 or bottom portion 16 or electrical connector 20) may be put into a mold and then overmolded with a low-pressure injection molding process.

Commonly used polymers for the encapsulation process include, e.g., polyurethane, silicone, epoxy resin. Commonly used polymers materials for this process include, e.g., Polyamide, Polyolefin, Polyurethane, Silicone, Epoxy Thermoset resin. The hotmelt adhesives or Epoxy Thermoset low viscosity resins have excellent adhesion to metals, PCB and electrical components, creating a water and dust tight encapsulation.

As is clear to a person of ordinary skill in the art, given the benefit of the present application, the electrical connector opening 56 of the enclosure 10 is designed to accommodate different types of connectors 20. Connectors 20 are provided with an adapter flange 24 which is preferably integrally molded around the perimeter of the connector housing. The adapter flange 24 for the various different connectors 20 has a common configuration which is designed to mate with the electrical connector opening 56 of the enclosure 10 Thus, this robust design can easily accommodate various different connector types such as Mate N Lok connectors with different pin configurations or MCON connectors with different pin configurations.

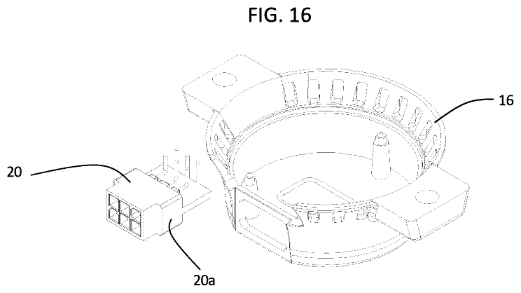

Alternatively, FIG. 16 shows that a variance 20a of electrical connector 20 may be overmolded to top portion 14, thereby integrating the connector variance with the top portion. The connector variances 20a may be either a standard connector or a custom injection-molded connector. The connector adapter flange seam 74 and the enclosure seam 62 advantageously seal the inside of the enclosure 10 from the deleterious external environment.

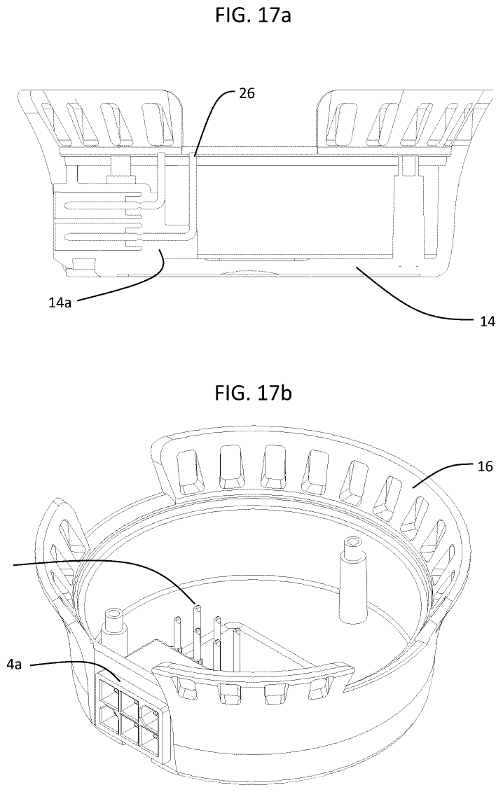

According to a further embodiment, FIG. 17a is a schematic side view of an embodiment of a gas detection sensor arrangement, with the PCBA and the bottom portion removed, showing connector pins 26 insert molded 14a into the top portion 14. FIG. 17b is a bottom perspective view of a gas detection sensor arrangement according to the embodiment of FIG. 17a. This embodiment further streamlines the assembly process and robustly protects the PCBA within the enclosure from dust, water or other contaminants.

The enclosure 10 may be designed to comply with Ingress Protection (IP) ratings. Ingress Protection (IP) testing assesses how well a product's enclosure can withstand dust, water, and other external elements. More specifically, the first numeral indicates the level of protection from solid objects (for example, dust) and is rated on a scale from 0 to 6. The second number indicates the level of protection from fluid or water provided by the enclosure and uses a scale from 0 to 9.

Depending on the IP rating desired, seams 62 and 74, may be joined with screws 27, 17 or snap-fit elements 77, 79 and may optionally include a sealing member 25, 75 mounted or disposed between the opposing surfaces of the seams 62, 74 and compressed when screws 27, 17 are tightened or snap-fit elements 77, 79 are engaged. Seams 62 and 74 may optionally be joined by adhesive bonding, e.g., UV-cured adhesives. By utilizing ultrasonic welding or laser welding instead of mechanical fasteners, the IP rating may be increased, for example, from IP54 to IP 66/67. Overmolding may increase the IP rating even further, for example, above IP 69. Thus, the enclosure 10 may be sealed to protect the interior electronics from dust and may even be dust tight. Enclosure 10 may also be sealed to protect the interior electronics from dripping water, sprayed or splashed water, or even from water jets (including high-pressure water jets).

This increased sealing effectiveness further makes the enclosure 10 mechanically robust as it provides protection against frost formation on the gas sensor 54 and against pressurized water jets when the enclosure 10 is being cleaned. This increased sealing effectiveness also eliminates the need for costly conformal coating on the circuit board 32 and the sensor PCBA 34, resulting in reduced costs for producing the gas detection sensor arrangement 100.

Another advantage of ultrasonic welding or laser welding is that assembling of the enclosure 10 can be automated, making the assembly process more accessible and production more scalable, which reduces the overall cost of manufacturing the gas detection sensor arrangement 100. The streamlined design of the enclosure 10 advantageously reduces the amount of time it takes to make each unit.

It should be noted that the terms, such as “comprising,” “including” or “having,” should be understood as not excluding other elements or steps and the words “a” or “an” should be understood as not excluding plurals of the elements or steps.

While the present disclosure has been illustrated and described with respect to one or more particular embodiments thereof, it should be appreciated by those of ordinary skill in the art that various modifications to this disclosure may be made without departing from the spirit and scope of the present disclosure.

1. A gas detection sensor arrangement comprising:
an enclosure having a top portion and a bottom portion;
a circuit board disposed within the enclosure; and
a gas sensor disposed within the enclosure, the gas sensor configured to detect gas in an environment external to the enclosure;
wherein the bottom portion and the top portion are joined together to seal the circuit board within the enclosure.

2. The gas detection sensor arrangement according to claim 1, wherein the bottom portion and the top portion are joined together using ultrasonic welding or laser welding.

3. The gas detection sensor arrangement according to claim 1, wherein the bottom portion and the top portion are joined together using mechanical fasteners, and wherein a sealing element is located between the bottom portion and the top portion.

4. The gas detection sensor arrangement according to claim 3, wherein the bottom portion and the top portion are joined using complementary snap-fit elements.

5. The gas detection sensor arrangement according to claim 1, wherein the bottom portion and the top portion are joined together using adhesive bonding.

6. The gas detection sensor arrangement according to claim 1, wherein the enclosure includes an electrical connector opening and an electrical connector having an electrical connector adapter flange, wherein the electrical connector opening and the electrical connector adapter flange have opposing surfaces joined together to seal the electrical connector opening.

7. The gas detection sensor arrangement according to claim 6, wherein the electrical connector opening and the electrical connector adapter flange are joined together using ultrasonic welding or laser welding.

8. The gas detection sensor arrangement according to claim 6, wherein the electrical connector opening and the electrical connector adapter flange are joined together using mechanical fasteners, and wherein a sealing element is located between the electrical connector opening and the electrical connector adapter flange.

9. The gas detection sensor arrangement according to claim 8, wherein the mechanical fasteners include complementary snap-fit elements.

10. The gas detection sensor arrangement according to claim 6, wherein the electrical connector opening and the electrical connector adapter flange are joined together using adhesive bonding.

11. The gas detection sensor arrangement according to claim 1, wherein the enclosure includes a gas sensor opening configured to allow gas in an environment external to the enclosure to reach the gas sensor, and wherein the enclosure biases the gas sensor towards the gas sensor opening to thereby seal the gas sensor opening.

12. The gas detection sensor arrangement according to claim 1, wherein one more microcontrollers, relays and/or switches are disposed on a first side of the circuit board and wherein the first side of the circuit board faces the top portion, wherein a gas sensor opening configured to allow gas in an environment external to the enclosure to reach the gas sensor is provided in the bottom portion of the enclosure, and wherein the gas sensor seals the gas sensor opening.

13. The gas detection sensor arrangement according to claim 1, further including a sealing member, wherein the enclosure includes a gas sensor opening configured to allow gas in an environment external to the enclosure to reach the gas sensor, and wherein the enclosure biases the gas sensor towards the gas sensor opening to thereby compress the sealing member between the gas sensor and the enclosure to seal the gas sensor opening.

14. The gas detection sensor arrangement according to claim 1, comprising a light guide to emit visible light signals wherein the light guide seals a light guide opening in the enclosure.

15. The gas detection sensor arrangement according to claim 1, wherein at least a portion of a wall of the enclosure is translucent or transparent to thereby allow light emitted by a light source located within the enclosure to be visible external to the enclosure.

16. The gas detection sensor arrangement according to claim 1, wherein the bottom portion includes an opening having an edge contour, wherein the gas sensor protrudes through the opening, and wherein the opening is sealed using a sealant between the edge contour and the gas sensor.

17. The gas detection sensor arrangement according to claim 1, wherein the circuit board and a connector and/or a cable are low-pressure encapsulated.

18. The gas detection sensor arrangement according to claim 6,
wherein the electrical connector opening and the electrical connector adapter flange are joined together using laser welding using a laser beam and wherein one of the top portion or the bottom portion are provided with a material allowing for the transmission of the laser beam energy and wherein the other of the top portion or the bottom portion are provided with a material allowing for the absorption of the laser beam energy, and/or
wherein the electrical connector opening and the electrical connector adapter flange are joined together using laser welding using a laser beam and wherein one of the electrical connector opening and the electrical connector adapter flange are provided with a material allowing for the transmission of the laser beam energy and wherein the other of the electrical connector opening and the electrical connector adapter flange are provided with a material allowing for the absorption of the laser beam energy.

19. The gas detection sensor arrangement according to claim 1, wherein at least one of the top portion and the bottom portion are provided with ribs.

20. The gas detection sensor arrangement according to claim 1, wherein at least one of the top portion, the bottom portion and an electrical connector housing include a UV-resistant polymer.

21. The gas detection sensor arrangement according to claim 1, wherein at least one of the top portion, the bottom portion and an electrical connector housing include a flame-retardant material with a minimum of UL94 flammability rating of V0.

22. A method for assembling a gas detection sensor arrangement, the method comprising:
providing a first portion of an enclosure for the gas detection sensor arrangement;
providing a second portion of the enclosure for the gas detection sensor arrangement;
providing an electrical connector having an electrical connector adapter flange;
joining the electrical connector adapter flange to an electrical connector opening provided in one of the first portion or the second portion;
assembling a circuit board having a gas sensor disposed thereon to the electrical connector; and
joining the first portion to the second portion at an enclosure seam to seal the circuit board inside the enclosure.

23. The method according to claim 22, wherein joining the first portion to the second portion includes using ultrasonic welding or laser welding.

24. The method according to claim 22, wherein joining the first portion to the second portion includes using mechanical fasteners and further includes providing a sealing element between the first portion and the second portion, wherein the sealing element is provided manually during assembly or is provided integrally with at least one of the first portion and the second portion.

25. The method according to claim 24, wherein the first portion is joined to the second portion using complementary snap-fit elements.

26. The method according to claim 22, wherein joining the first portion to the second portion includes using ultrasonic welding and providing an energy director located on one of the first portion or the second portion to facilitate the ultrasonic welding, or wherein joining the first portion to the second portion includes using laser welding and providing a shear joint.

27. The method according to claim 22, the method further comprising:
placing a sealing member between the gas sensor and one of the first portion or the second portion; and
compressing the sealing member between the gas sensor and the one of the first portion or the second portion during the step of joining the first portion to the second portion.

28. The method according to claim 27, wherein the step of placing the sealing member between the gas sensor and the one of the first portion or the second portion includes placing the sealing member around a gas sensor opening formed in the one of the first portion or the second portion, and wherein the step of compressing the sealing member between the gas sensor and the one of the first portion or the second portion during the step of joining the first portion to the second portion seals the gas sensor opening.

29. The method according to claim 22, wherein the step of joining the electrical connector adapter flange to the electrical connector opening includes using ultrasonic welding or laser welding.

30. The method according to claim 22, wherein the step of joining the electrical connector adapter flange to the electrical connector opening includes using mechanical fasteners, and further includes locating and compressing a sealing element between the first portion and the bottom portion.

31. The method according to claim 30, wherein the electrical connector adapter flange is joined to the electrical connector opening using complementary snap-fit elements.

32. The method according to claim 22,
wherein the step of joining the first portion to the second portion includes using ultrasonic welding and further includes providing an energy director between opposing surfaces of the first portion and the second portion, at least partially melting the energy director, and bringing the opposing surfaces of the first portion and the second portion together at the enclosure seam as the energy director melts, and/or
wherein the step of joining the electrical connector adapter flange to the electrical connector opening includes using ultrasonic welding and further includes providing an energy director between opposing surfaces of the electrical connector adapter flange and the electrical connector opening, at least partially melting the energy director, and bringing the opposing surfaces of the electrical connector adapter flange and the electrical connector opening together as the energy director melts.

33. The method according to claim 22,
wherein the step of joining the first portion to the second portion includes using laser welding using a laser beam, providing one of the top portion or the bottom portion with a material allowing for the transmission of the laser beam energy, and providing the other of the top portion or the bottom portion with a material allowing for the absorption of the laser beam energy, and/or
wherein the step of joining the electrical connector opening and the electrical connector adapter flange includes using laser welding using a laser beam, providing one of the electrical connector opening and the electrical connector adapter flange with a material allowing for the transmission of the laser beam energy, and providing the other of the electrical connector opening and the electrical connector adapter flange with a material allowing for the absorption of the laser beam energy.

34. The gas detection sensor arrangement according to claim 1, wherein the circuit board and a connector and/or a cable are low-pressure encapsulated within enclosure.
